(19) United States
(12) Patent Application Publication
(10) Pub. No.: US 2026/0102179 A1
(43) Pub. Date: Apr. 16, 2026

(54) MEDICAL DEVICE

(71) Applicant: TERUMO KABUSHIKI KAISHA, Tokyo (JP)

(72) Inventor: Yusaku Yamaki, Kanagawa (JP)

(21) Appl. No.: 19/407,424

(22) Filed: Dec. 3, 2025

Related U.S. Application Data
(63) Continuation of application No. PCT/JP2024/020844, filed on Jun. 7, 2024.

(30) Foreign Application Priority Data
Jun. 9, 2023 (JP) 2023095444

(51) Int. Cl. A61B 17/295; A61B 17/00; A61B 17/29; A61B 18/00; A61B 18/14

(52) U.S. Cl. CPC A61B 17/295; A61B 18/1447; A61B 2017/00778; A61B 2017/2944; A61B 2018/00428; A61B 2018/00595; A61B 2018/1455

(57) ABSTRACT

A medical device (10) includes a first jaw (32a) and a second jaw (32b). A first proximal end portion (38a) of the first jaw has a first protrusion (72a), and a second proximal end portion (38b) of the second jaw has a second protrusion (72b). When the first jaw and the second jaw are in a closed state, the first protrusion protrudes toward the second proximal end portion, and the second protrusion protrudes toward the first proximal end portion. The first protrusion has a first sliding contact surface (78a) that comes into sliding contact with the second proximal end portion when the first jaw and the second jaw are closed. The second protrusion has a second sliding contact surface (78b) that comes into sliding contact with the first proximal end portion when the first jaw and the second jaw are closed.

### CROSS REFERENCE TO RELATED APPLICATIONS

This application is a continuation of PCT Application No. PCT/JP2024/020844, filed Jun. 7, 2024, based on and claiming priority to Japanese Application No. JP2023095444, Filed Jun. 9, 2023, both of which are incorporated herein by reference in their entirety.

### BACKGROUND OF THE INVENTION

The present invention relates to a medical device for pinching a piece of biological tissue between a first jaw and a second jaw that are openable and closable by bringing both the jaws in a closed state.

In coronary artery bypass surgery, a blood vessel bypassing a lesion site is connected to an artery. The blood vessel is harvested from, for example, a lower limb of a patient by an endoscopic vessel harvesting system.

The endoscopic vessel harvesting system includes a medical device including a vessel harvesting tool. As a medical device, a configuration having a first jaw and a second jaw that can be opened and closed is conceivable as described in Japanese Patent Application Laid-Open No. 2011-229923. In this configuration, first, the first jaw and the second jaw are changed from an open state to a closed state inside the body of a patient to pinch a blood vessel between the first jaw and the second jaw. Next, the blood vessel is cut with a cutter. A vessel harvesting tool is moved to the outside of the patient's body, whereby the blood vessel pinched between the first jaw and the second jaw is extracted outside the body. In this case, the biological tissue harvested by the medical device is a blood vessel.

### SUMMARY OF THE INVENTION

If the axis of the first jaw and the axis of the second jaw are misaligned when the first jaw and the second jaw are in the closed state, it is not easy to satisfactorily hold biological tissue such as a blood vessel with the first jaw and the second jaw.

It is therefore an object of the present invention to solve the above-described problems.

A first aspect of the present invention provides a medical device including a first jaw and a second jaw that are openable and closable by rotation and that pinch biological tissue when in a closed state, wherein the first jaw has a first pinching portion and a first proximal end portion connected to a proximal side of the first pinching portion, the second jaw has a second pinching portion that pinches the biological tissue together with the first pinching portion, and a second proximal end portion connected to a proximal side of the second pinching portion, the first proximal end portion and the second proximal end portion have a first protrusion and a second protrusion, respectively, the first protrusion protruding toward the second proximal end portion, and the second protrusion protruding toward the first proximal end portion, when the first jaw and the second jaw are in the closed state, the first protrusion has a first sliding contact surface that comes into sliding contact with the second proximal end portion when the first jaw and the second jaw are closed, and the second protrusion has a second sliding contact surface that comes into sliding contact with the first proximal end portion when the first jaw and the second jaw are closed. Note that the biological tissue in the present invention includes a biological organ that is an aggregate of pieces of biological tissue.

With this configuration, when the first jaw and the second jaw are closed, a state is formed in which the first sliding contact surface of the first protrusion comes into contact with the second proximal end portion and the second sliding contact surface of the second protrusion comes into contact with the first proximal end portion. Therefore, when the first jaw and the second jaw are in the closed state, it is easy to set the axis of the first pinching portion and the axis of the second pinching portion to be parallel to and face each other. In other words, when the first jaw and the second jaw are in the closed state, it is possible to suppress axial deviation between the first pinching portion and the second pinching portion. Therefore, it is possible to satisfactorily hold the biological tissue by the first pinching portion and the second pinching portion.

In the medical device according to the first aspect of the present invention, the first proximal end portion and the second proximal end portion may respectively have a first flat surface and a second flat surface facing each other when the first jaw and the second jaw are in the closed state, the first protrusion may protrude from the first flat surface toward the second flat surface, and the second protrusion may protrude from the second flat surface toward the first flat surface.

In this configuration, when the first jaw and the second jaw are closed, the first flat surface and the second flat surface gradually face each other. Therefore, the first sliding contact surface provided on the first protrusion easily comes into contact with the second flat surface (second proximal end portion), and the second sliding contact surface of the second protrusion easily comes into contact with the first flat surface (first proximal end portion).

In the medical device according to an aspect of the present invention, the first pinching portion may have a first cutter groove extending along a direction of the axis of the first pinching portion, and the second pinching portion may have a second cutter groove extending along a direction of the axis of the second pinching portion, the medical device further including a cutter that, when the first jaw and the second jaw are in the closed state, may advance toward distal ends of the first jaw and the second jaw to be inserted into the first cutter groove and the second cutter groove, and further advance toward the distal ends of the first jaw and the second jaw along the first cutter groove and the second cutter groove.

When the cutter moves from the proximal end to the distal end along the first cutter groove and the second cutter groove, the cutter cuts the biological tissue. As described above, the axial deviation between the first pinching portion and the second pinching portion is suppressed, and thus, when the first jaw and the second jaw are in the closed state, relative misalignment between the first cutter groove and the second cutter groove is minimized. Therefore, the cutter is easily inserted into the first cutter groove and the second cutter groove, and easily moves along the first cutter groove and the second cutter groove.

In the medical device according to above, the first protrusion may have a first linear portion extending along a direction of the axis of the first proximal end portion, the second protrusion may have a second linear portion extending along a direction of the axis of the second proximal end portion, and the first linear portion and the second linear portion may be positioned parallel to each other when the first jaw and the second jaw are in the closed state.

When the cutter moves in the closed state of the first jaw and the second jaw, the first linear portion and the second linear portion are parallel to each other, whereby the cutter is prevented from interfering with the first linear portion and the second linear portion. Therefore, even when the first jaw and the second jaw are in the closed state, the cutter easily moves.

In the medical device according to above, the first linear portion may be located proximal to a rotation center of the first jaw, and the second linear portion may be located proximal to a rotation center of the second jaw.

With this configuration, when the first jaw and the second jaw are changed from the open state to the closed state, the first proximal end portion can be supported by the second sliding contact surface of the second linear portion while the second proximal end portion is supported by the first sliding contact surface of the first linear portion. Therefore, it is possible to further suppress the axial deviation between the first pinching portion and the second pinching portion.

In the medical device according to above, the first protrusion may have a first inclined portion connected to a distal side of the first linear portion, and the second protrusion may have a second inclined portion connected to a distal side of the second linear portion, the first inclined portion and the second inclined portion being inclined in a direction not interfering with the cutter when the first jaw and the second jaw are in an open state.

In the open state of the first jaw and the second jaw, the first inclined portion and the second inclined portion serve as back clearance portions that do not interfere with the distal end of the cutter. Therefore, even if the first jaw and the second jaw are in the open state when the cutter is at the most retracted position, the first protrusion and the second protrusion are prevented from interfering with the cutter. Moreover, in this case, the sliding contact area of the first sliding contact surface and the second sliding contact surface when the first jaw and the second jaw are changed from the open state to the closed state is larger than that when the first inclined portion and the second inclined portion are not provided. Therefore, it is possible to further suppress the axial deviation between the first pinching portion and the second pinching portion when the first jaw and the second jaw are changed from the open state to the closed state.

In the medical device according to any of the above aspects of the present invention, the first proximal end portion may have a first extension-side end portion that is an end portion having a distal end from which the first pinching portion extends in a distal direction, and a first non-extension-side end portion that is an end portion opposite to the first extension-side end portion with reference to a first parallel line passing through a rotation center of the first jaw and parallel to an axis of the first jaw, the second proximal end portion may have a second extension-side end portion that is an end portion having a distal end from which the second pinching portion extends in the distal direction, and a second non-extension-side end portion that is an end portion opposite to the second extension-side end portion with reference to a second parallel line passing through a rotation center of the second jaw and parallel to an axis of the second jaw, the first protrusion may be located at the first non-extension-side end portion, and the second protrusion may be located at the second non-extension-side end portion.

With this configuration, when the first jaw and the second jaw are changed from the open state to the closed state, the first proximal end portion can be supported by the second linear portion while the second proximal end portion is supported by the first linear portion. In addition, when the medical device includes a cutter, it is possible to reliably avoid the first inclined portion and the second inclined portion from interfering with the cutter when the first jaw and the second jaw are in the open state.

According to an embodiment of the present invention, when the first jaw and the second jaw are in the closed state, it is easy to set the axis of the first pinching portion and the axis of the second pinching portion to be parallel to and face with other. In other words, when the first jaw and the second jaw are in the closed state, it is possible to suppress axial deviation between the first pinching portion and the second pinching portion. Therefore, it is possible to satisfactorily hold the biological tissue by the first pinching portion and the second pinching portion.

### BRIEF DESCRIPTION OF THE DRAWINGS

FIG. 1 is a perspective view of a main part of a medical device.

FIG. 2 is a perspective view of the main part of the medical device without illustrating a first cylindrical body.

FIG. 3 is an exploded perspective view of a harvesting tool constituting the medical device.

FIG. 4 is a rear view of a main part of a first jaw and a second jaw as viewed from a proximal side toward a distal side.

FIG. 5 is a schematic cross-sectional side view of the harvesting tool in a closed state.

FIG. 6 is a schematic plan view of a main part of the harvesting tool in a closed state.

FIG. 7 is a schematic cross-sectional side view illustrating a state in which a cutter advances in the harvesting tool in the closed state.

FIG. 8 is a schematic cross-sectional side view of the harvesting tool in an open state.

### DETAILED DESCRIPTION OF PREFERRED EMBODIMENTS

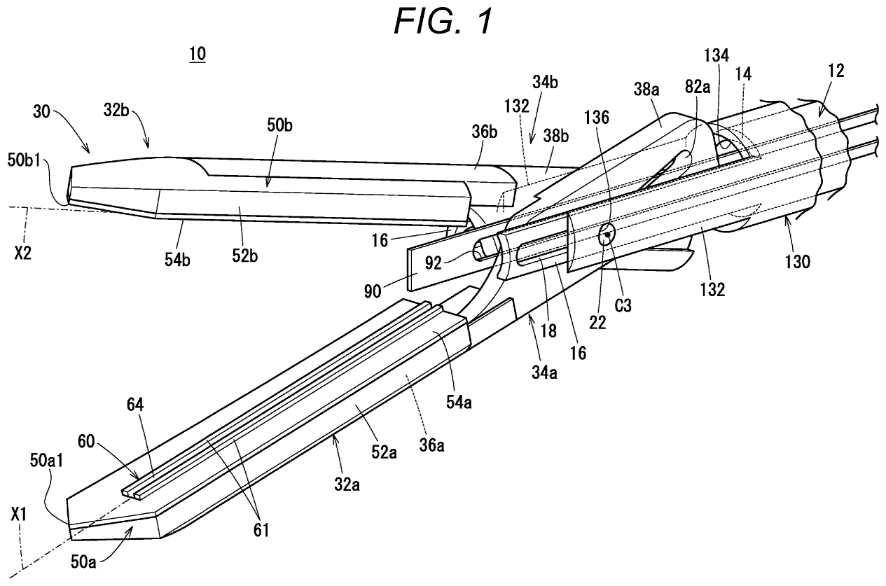

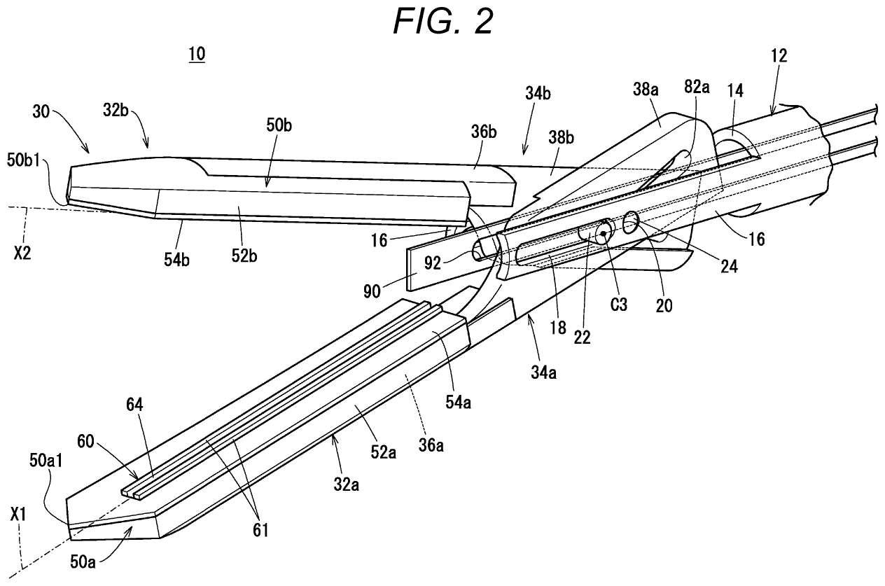

In the following description, the term “distal end” refers to, for each component, a direction approaching biological tissue when the biological tissue is harvested. The term “proximal end” refers to, for each component, an opposite direction to the distal end. For example, in a first jaw 32a illustrated in FIGS. 1 and 2, a first pinching portion 50a is positioned relatively on a distal side with respect to a first proximal end portion 38a. The first proximal end portion 38a is positioned relatively on a proximal side with respect to the first pinching portion 50a.

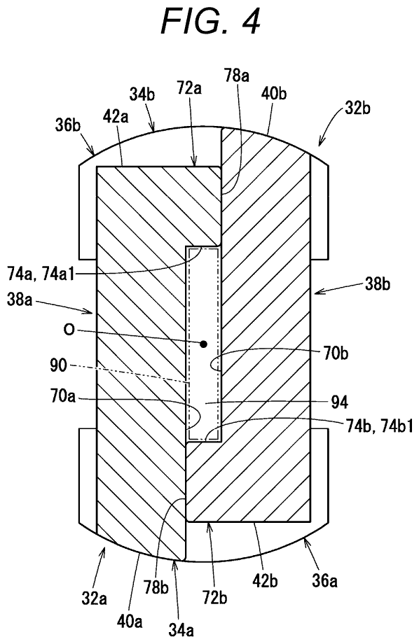

The position and the shape of the first jaw 32a are rotationally symmetric with the position and the shape of the second jaw 32b about a symmetry center O illustrated in FIG. 4. Therefore, for names of components of the second jaw 32b, which are identical to components of the first jaw 32a, the term “first” in the name of each of the components of the first jaw 32a is replaced with the term “second” for components of the second jaw 32b. In addition, for the components of the second jaw 32b, which are identical to the components of the first jaw 32a, the alphabet “a” in a reference sign of each of the components of the first jaw 32a is in principle replaced with “b” in a reference sign of each of the components of the second jaw 32b. Note that detailed description of the components of the second jaw 32b may be omitted.

The wording “opening and closing of a harvesting tool 30” is synonymous with “opening and closing of the first jaw 32a and the second jaw 32b”. The wording “the harvesting tool 30 is in an open state” is synonymous with “the first jaw 32a and the second jaw 32b are in the open state,” and “the harvesting tool 30 is in a closed state” is synonymous with “the first jaw 32a and the second jaw 32b are in the closed state.”

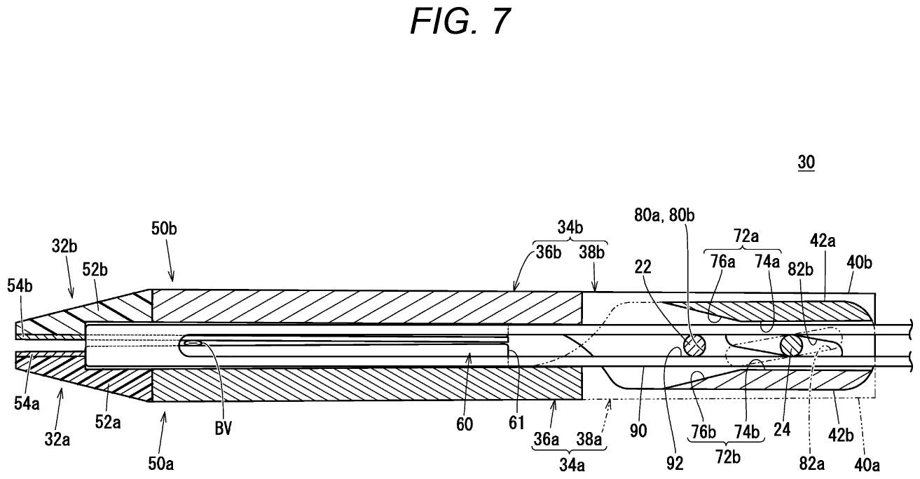

A case where a blood vessel (for example, a branch vessel BV illustrated in FIG. 7) is a piece of biological tissue that is an object to be harvested will now be described as an example. However, the object to be harvested is not limited to a blood vessel. The object to be harvested may be a body lumen of a living body, other than a blood vessel. Note herein that biological tissue referred to in the present invention includes biological organs. Examples of the biological organs include a luminal organ. Examples of luminal organs include a digestive tract, a ureter, a trachea, and a cervical canal.

FIG. 1 is a perspective view of a main part of a medical device 10 according to the present embodiment. The medical device 10 includes a first cylindrical body 130, a second cylindrical body 12, and a harvesting tool 30. The first cylindrical body 130 and the second cylindrical body 12 are cylindrical members linearly extending in the direction of axis. In the illustrated example, the first cylindrical body 130 and the second cylindrical body 12 have a cylindrical shape, but may have a polygonal cylindrical shape.

The first cylindrical body 130 includes a pair of first extension portions 132. The pair of first extension portions 132 extends from the distal end of the first cylindrical body 130 along the direction of an axis of the first cylindrical body 130 extending in a proximal to distal direction. A pair of first cutout grooves 134 is formed between the pair of first extension portions 132. The pair of first cutout grooves 134 is disposed at positions separated away from each other by an angle of 180° in the circumferential direction. The pair of first cutout grooves 134 extends in the direction of the axis of the first cylindrical body 130.

A pair of support holes 136 is formed in the pair of first extension portions 132, respectively. The directions of axes of the support holes 136 are orthogonal to the direction of the axis of the first cylindrical body 130. Each of the support holes 136 penetrates from the outer surface to the inner surface of the corresponding first extension portion 132. The center positions of the pair of support holes 136 are shifted by 90° in the circumferential direction of the first cylindrical body 130 with respect to the centers of the pair of first cutout grooves 134. Both end portions of a rotation shaft 22 in the direction of an axis described later are fitted into the pair of support holes 136, respectively. Based on this fitting, the rotation shaft 22 is positioned and fixed with respect to the first cylindrical body 130.

Although not illustrated, a proximal end portion of the first cylindrical body 130 is provided with, for example, a jaw operation unit, a cutter operation unit, and an energization switch. For example, a wire (not illustrated) through which a high-frequency current flows is accommodated in the hollow interior of the first cylindrical body 130.

The second cylindrical body 12 is shorter than the first cylindrical body 130 and is accommodated in the hollow interior at the distal end of the first cylindrical body 130. As an operator operates the jaw operation unit, power is transmitted to the second cylindrical body 12 via a power transmission member (not illustrated). As a result, the second cylindrical body 12 moves inside the first cylindrical body 130. In this manner, the second cylindrical body 12 is movably accommodated inside the first cylindrical body 130.

As illustrated in FIG. 2, which does not illustrate the first cylindrical body 130, the second cylindrical body 12 has a pair of second extension portions 16. The pair of second extension portions 16 extends from a distal end of the second cylindrical body 12 in the direction of the axis of the second cylindrical body 12. A pair of second cutout grooves 14 is formed between the pair of second extension portions 16. The pair of second cutout grooves 14 is formed at positions separated away from each other by an angle of 180° in the circumferential direction. The pair of second cutout grooves 14 extends in the direction of the axis of the second cylindrical body 12. The pair of first extension portions 132 covers the pair of second extension portions 16. The pair of second cutout grooves 14 overlaps the pair of first cutout grooves 134.

The pair of second extension portions 16 is formed with a pair of guide grooves 18 and a pair of pin attachment holes 20, respectively. The pair of guide grooves 18 extends in the direction of the axis of the second cylindrical body 12 and fully penetrates from the outer surfaces to the inner surfaces of the pair of second extension portions 16. The pair of pin attachment holes 20 is positioned proximal to the pair of guide grooves 18. Each of the pair of pin attachment holes 20 is a circular through hole penetrating from the outer surface to the inner surface of the corresponding one of the pair of second extension portions 16. The center positions of the pair of guide grooves 18 and the pair of pin attachment holes 20 are shifted from the center of the pair of second cutout grooves 14 by 90° in the circumferential direction of the second cylindrical body 12.

The rotation shaft 22 having a columnar shape is inserted into the pair of guide grooves 18. The rotation shaft 22 moves relative to the second cylindrical body 12 in the guide grooves 18 along with displacement of the harvesting tool 30 in the direction of the axis. In other words, the guide grooves 18 allow relative movement of the second cylindrical body 12 with respect to the rotation shaft 22. Note that, as described above, the end portions of the rotation shaft 22 are fitted into the pair of support holes 136 formed in the pair of first extension portions 132 of the first cylindrical body 130.

End portions of an opening and closing pin 24 are inserted into the pair of pin attachment holes 20. The opening and closing pin 24 is a pin fixed to the second cylindrical body 12. When the harvesting tool 30 is displaced in the direction of the axis, the opening and closing pin 24 is displaced relative to the first jaw 32a and the second jaw 32b.

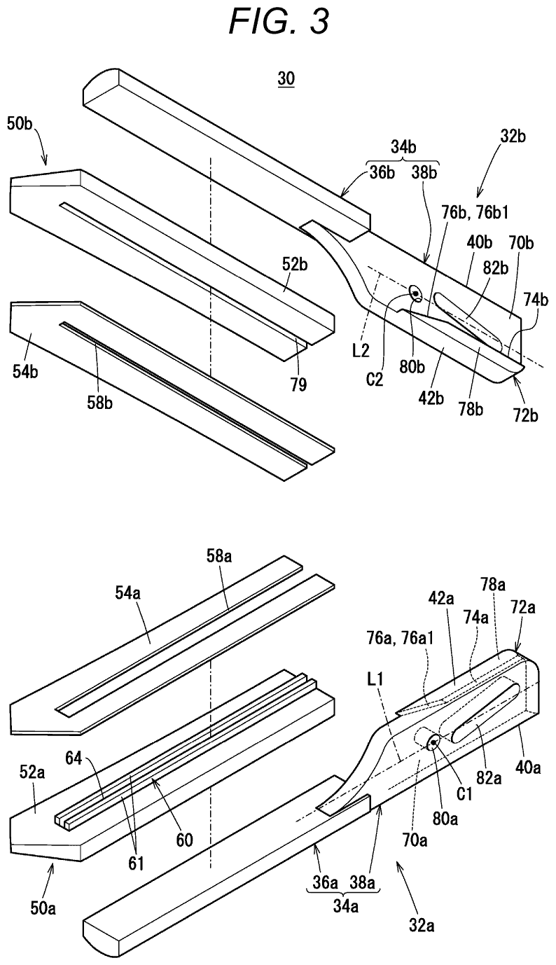

The harvesting tool 30 includes the first jaw 32a, the second jaw 32b, and a cutter 90. As illustrated in FIG. 3, the first jaw 32a includes a first base member 34a. The first base member 34a integrally includes a first support portion 36a having a flat surface and a curved surface, and a first proximal end portion 38a connected to the first support portion 36a. The material of the first base member 34a is typically metal.

The first support portion 36a is positioned toward a distal end of the first base member 34a. The first proximal end portion 38a is positioned toward a proximal end of the first base member 34a. The thickness direction of the first support portion 36a is orthogonal to the thickness direction of the first proximal end portion 38a. The width direction of the first support portion 36a (direction orthogonal to a first parallel line L1 and the thickness direction of the first support portion 36a) is parallel to the thickness direction of the first proximal end portion 38a (direction orthogonal to the first parallel line L1 and the thickness direction of the first proximal end portion 38a). In the following description, an end (lower end in FIG. 3) of the first proximal end portion 38a from which the first support portion 36a extends is referred to as a first extension-side end portion 40a. That is, the first support portion 36a extends in the distal direction from the distal end of the first extension-side end portion 40a of the first proximal end portion 38a.

An end (upper end in FIG. 3) opposite to the first extension-side end portion 40a is referred to as a first non-extension-side end portion 42a. The first non-extension-side end portion 42a is positioned on the opposite side of the first extension-side end portion 40a based on the first parallel line L1 passing through a first bearing hole 80a and parallel to an axis of the first jaw 32a. Note that a center C1 of the first bearing hole 80a is the rotation center of the first jaw 32a.

The first support portion 36a constitutes the first pinching portion 50a. Specifically, the first pinching portion 50a includes the first support portion 36a, a plate-shaped first main body 52a made of an insulator, and a first electrode 54a. The first main body 52a and the first electrode 54a are supported by the first support portion 36a in a stacked state. The first main body 52a is interposed between the first support portion 36a and the first electrode 54a, and electrically insulates the first support portion 36a from the first electrode 54a.

The first main body 52a includes a spacer 60 protruding toward a second support portion 36b. The spacer 60 may be formed integrally with the first main body 52a or may be a separate member from the first main body 52a. The spacer 60 includes a pair of side walls 61. A first cutter groove 64 is formed between the pair of side walls 61. The first cutter groove 64 extends in the direction of the axis of the first jaw 32a and guides the movement of the cutter 90 in the direction of the axis. The width of the first cutter groove 64 is equal to or slightly larger than the thickness (size in the width direction) of the cutter 90. The pair of side walls 61 is present between the cutter 90 inserted into the first cutter groove 64 and the first electrode 54a to electrically insulate the cutter 90 and the first electrode 54a from each other.

The first electrode 54a is formed with a first slit 58a. The pair of side walls 61 of the spacer 60 is exposed from the first slit 58a and protrudes toward the second support portion 36b.

The first pinching portion 50a configured as described above has a first axis X1. A most distal end 50a1 of the first pinching portion 50a may be positioned on the first axis X1 or may be shifted from the first axis X1.

The first proximal end portion 38a has a flange shape wider than the first support portion 36a. The first proximal end portion 38a has a first flat surface 70a. The first flat surface 70a includes a first protrusion 72a protruding in a direction orthogonal to the axis of the first proximal end portion 38a (or the axis of the first jaw 32a). The first protrusion 72a is provided on the first non-extension-side end portion 42a of the first proximal end portion 38a. The first protrusion 72a includes a first linear portion 74a and a first inclined portion 76a. The first linear portion 74a extends linearly along the axis from the proximal end toward the distal end of the first proximal end portion 38a. The first linear portion 74a includes a plane surface 74a1 (see FIG. 4) having a width parallel to the center C1 of the first bearing hole 80a. The plane surface 74a1 extends parallel to the symmetry center O. The width of the plane surface 74a1 corresponds to a protrusion amount of the first protrusion 72a. The first inclined portion 76a is inclined in a direction away from the first parallel line L1 as the first inclined portion 76a extends to the distal end of the first proximal end portion 38a. The first inclined portion 76a is defined by a plane surface 76a1 (see FIG. 3) having a width parallel to the center C1 of the first bearing hole 80a. The width of the plane surface 76a1 corresponds to the protrusion amount of the first protrusion 72a.

The first protrusion 72a may be provided on the first extension-side end portion 40a of the first proximal end portion 38a. The first protrusion 72a may be provided at both the first extension-side end portion 40a and the first non-extension-side end portion 42a of the first proximal end portion 38a. The first protrusion 72a and a second protrusion 72b may be provided such that the first non-extension-side end portion 42a of the first proximal end portion 38a and a second non-extension-side end portion 42b of a second proximal end portion 38b face each other. The first protrusion 72a having only the first linear portion 74a may be provided by cutting off the first inclined portion 76a. The same applies to the second protrusion 72b described later.

The first protrusion 72a includes a first sliding contact surface 78a. As will be understood from FIG. 4, when the harvesting tool 30 is in the closed state, the first sliding contact surface 78a faces the second proximal end portion 38b constituting the second jaw 32b. As illustrated in FIG. 3, the first bearing hole 80a and a first slide groove 82a are formed in the first proximal end portion 38a.

A second base member 34b of the second jaw 32b integrally includes the second support portion 36b and the second proximal end portion 38b. The second support portion 36b is positioned at a distal end of the second base member 34b. The second proximal end portion 38b is positioned at a proximal end of the second base member 34b. The thickness direction of the second support portion 36b is orthogonal to the thickness direction of the second proximal end portion 38b. The width direction of the second support portion 36b (direction orthogonal to a second parallel line L2 and the thickness direction of the second support portion 36b) is parallel to the thickness direction of the second proximal end portion 38b (direction orthogonal to the second parallel line L2 and the thickness direction of the second proximal end portion 38b). The second support portion 36b constitutes a second pinching portion 50b together with a second main body 52b and a second electrode 54b. The second main body 52b and the second electrode 54b are formed with a second cutter groove 79 and a second slit 58b, respectively. The second slit 58b overlaps the second cutter groove 79.

The second slit 58b is wider than the first cutter groove 64. Therefore, when the cutter 90 moves into the first cutter groove 64, side surfaces of the cutter 90 are prevented from coming into contact with inner surfaces of the second slit 58b. In other words, the side surfaces of the cutter 90 and side surfaces of the second slit 58b do not come in contact with each other. Due to such non-contact, the cutter 90 and the second electrode 54b are electrically insulated from each other.

The second pinching portion 50b has a second axis X2. A most distal end 50b1 of the second pinching portion 50b may be positioned on the second axis X2 or may be shifted from the second axis X2. In the latter case, it is preferable that a direction of deviation of the most distal end 50a1 of the first pinching portion 50a with respect to the first axis X1 and a direction of deviation of the most distal end 50b1 of the second pinching portion 50b with respect to the second axis X2 are set to be the same.

The second protrusion 72b provided on the second non-extension-side end portion 42b of the second proximal end portion 38b includes a second linear portion 74b, a second inclined portion 76b, and a second sliding contact surface 78b. The second linear portion 74b includes a plane surface 74b1 (see FIG. 4) having a width parallel to a center C2 of a second bearing hole 80b. The plane surface 74b1 extends parallel to the symmetry center O. The width of the plane surface 74b1 corresponds to a protrusion amount of the second protrusion 72b. The second inclined portion 76b is inclined in a direction away from the second parallel line L2 as the second inclined portion 76b extends to the distal end of the second proximal end portion 38b. The second inclined portion 76b is defined by a plane surface 76b1 (see FIG. 3) having a width parallel to the center C2 of the second bearing hole 80b. The width of the plane surface 76b1 corresponds to the protrusion amount of the second protrusion 72b.

As will be understood from FIG. 4, when the harvesting tool 30 is in the closed state, the second sliding contact surface 78b faces the first proximal end portion 38a. When the first jaw 32a and the second jaw 32b are closed, the first sliding contact surface 78a of the first protrusion 72a is in sliding contact with the second flat surface 70b of the second extension-side end portion 40b of the second proximal end portion 38b. Note herein that the second non-extension-side end portion 42b is positioned on an opposite side of the second extension-side end portion 40b based on the second parallel line L2 (see FIG. 3) passing through the second bearing hole 80b and parallel to the axis of the second jaw 32b. The second sliding contact surface 78b of the second protrusion 72b is in sliding contact with the first flat surface 70a of the first extension-side end portion 40a of the first proximal end portion 38a.

The first flat surface 70a and the second flat surface 70b are separated away from each other by a distance corresponding to the protrusion amount of each of the first protrusion 72a and the second protrusion 72b (see FIG. 4). Therefore, a cutter space 94 is formed by the first flat surface 70a, the first protrusion 72a, the second flat surface 70b, and the second protrusion 72b. Specifically, the cutter space 94 is formed by the first flat surface 70a, the plane surface 74b1 of the second linear portion 74b, the second flat surface 70b, and the plane surface 74a1 of the first linear portion 74a.

As illustrated in FIG. 3, the second bearing hole 80b and a second slide groove 82b are formed in the second proximal end portion 38b. As illustrated in FIG. 2, when the first proximal end portion 38a of the first jaw 32a and the second proximal end portion 38b of the second jaw 32b are inserted into the second cutout grooves 14 of the second cylindrical body 12 and held between the pair of second extension portions 16, the first bearing hole 80a and the second bearing hole 80b are located between the pair of guide grooves 18. Therefore, the rotation shaft 22 is allowed to pass through one of the pair of support holes 136, one of the pair of guide grooves 18, the first bearing hole 80a, an elongated hole 92 (described later) formed in the cutter 90, the second bearing hole 80b, the other of the pair of guide grooves 18, and the other of the pair of support holes 136. The rotation shaft 22 couples the first proximal end portion 38a of the first jaw 32a and the second proximal end portion 38b of the second jaw 32b to each other. The center C1 (see FIG. 3) of the first bearing hole 80a, a center C3 (see FIG. 2) of the rotation shaft 22, and the center C2 (see FIG. 3) of the second bearing hole 80b overlap each other.

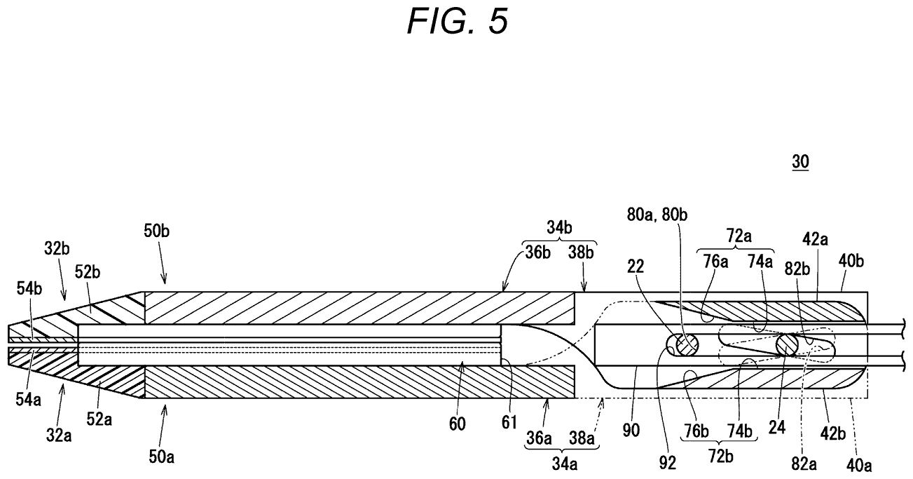

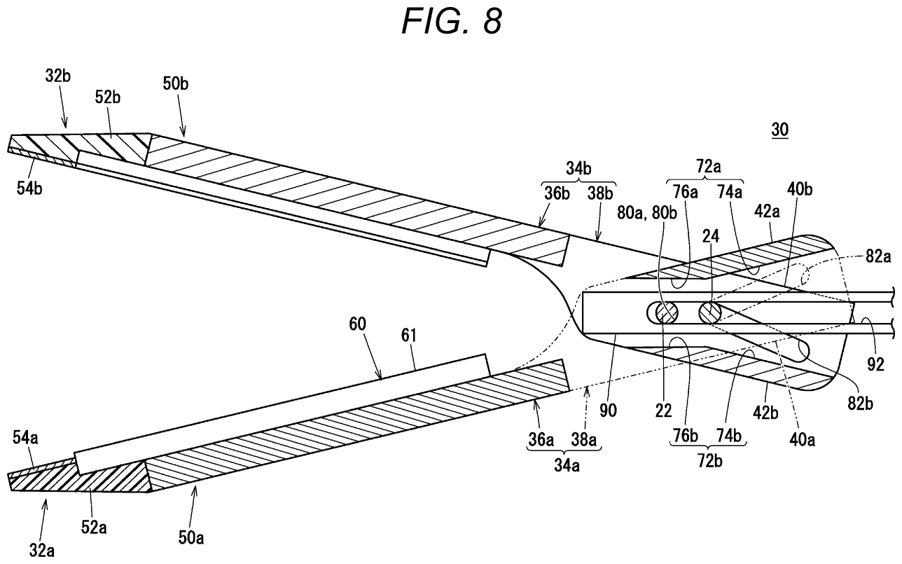

As illustrated in FIG. 5, the first slide groove 82a is positioned proximal to the first bearing hole 80a at the first proximal end portion 38a. The first slide groove 82a is inclined from the first extension-side end portion 40a toward the first non-extension-side end portion 42a as it extends from the distal end toward the proximal end. The same applies to the second slide groove 82b formed in the second proximal end portion 38b. The first jaw 32a and the second jaw 32b are rotationally symmetric, and thus, when the first jaw 32a and the second jaw 32b are in the open state, the first slide groove 82a and the second slide groove 82b form a V shape with its opening directed to the proximal end (see FIG. 8). On the other hand, when the first jaw 32a and the second jaw 32b are in the closed state, the first slide groove 82a and the second slide groove 82b intersect each other to form an X shape (see FIG. 5).

As illustrated in FIG. 2, when the first proximal end portion 38a of the first jaw 32a and the second proximal end portion 38b of the second jaw 32b are held between the pair of second extension portions 16 of the second cylindrical body 12, the pair of pin attachment holes 20 overlaps each other at predetermined positions of the first slide groove 82a and the second slide groove 82b. Therefore, the opening and closing pin 24 is allowed to pass through one of the pair of pin attachment holes 20, the first slide groove 82a, the elongated hole 92, the second slide groove 82b, and the other of the pair of pin attachment holes 20. As will be described later, as the opening and closing pin 24 moves relative to the first slide groove 82a and the second slide groove 82b, the first jaw 32a and the second jaw 32b are opened or closed.

The cutter 90 is disposed between the first proximal end portion 38a and the second proximal end portion 38b. The cutter 90 extends in the direction of axes of the first cylindrical body 130 and the second cylindrical body 12. The cutter 90 is positioned on the proximal side in an initial state. The cutter operation unit is provided on the proximal end portion (not illustrated) of the first cylindrical body 130, as described above. When the operator operates the cutter operation unit, the cutter 90 can move forward or backward in the distal direction or a proximal direction along the direction of the axis. Note that, when the harvesting tool 30 is in the closed state, the cutter 90 moves toward the distal end in the direction of the axis along the first cutter groove 64 in the cutter space 94 illustrated in FIG. 4.

As illustrated in FIG. 2, the cutter 90 has the elongated hole 92 penetrating in the thickness direction of the cutter 90. The rotation shaft 22 and the opening and closing pin 24 pass through the elongated hole 92.

The medical device 10 according to the present embodiment is basically configured as described above. Next, vessel harvesting using the medical device 10 will be described. Note that the operator in the following description is, for example, a medical worker, typically a doctor.

To harvest a blood vessel such as a branch vessel BV (see FIG. 7) from the body of a patient, the medical device 10 and an imaging device are inserted into the body of the patient. The medical device 10 is brought close to the branch vessel BV that is an object to be harvested, and the medical device 10 is brought into the open state illustrated in FIG. 8. In other words, the first pinching portion 50a of the first jaw 32a and the second pinching portion 50b of the second jaw 32b are separated away from each other. At this point, the harvesting tool 30 is positioned on the proximal side of the second cutout grooves 14 of the second cylindrical body 12. The operator places the harvesting tool 30 in the open state at a position near the predetermined branch vessel BV while performing observation with the imaging device.

Next, the operator operates the jaw operation unit described above. With this operation, the second cylindrical body 12 moves toward the proximal side inside the first cylindrical body 130.

The opening and closing pin 24 is immovable with respect to the second cylindrical body 12. Therefore, the opening and closing pin 24 moves relative to the first slide groove 82a of the first jaw 32a and the second slide groove 82b of the second jaw 32b. The relative movement direction of the opening and closing pin 24 is toward the proximal side of the first jaw 32a and the second jaw 32b. In the open state of the harvesting tool 30, the first slide groove 82a and the second slide groove 82b form a V shape with its opening directed to the proximal end. The opening and closing pin 24 before the movement is located at the vertex of the V shape. Therefore, as the opening and closing pin 24 moves to the proximal side relative to the first slide groove 82a and the second slide groove 82b, the first jaw 32a and the second jaw 32b rotate about the center C3 of the rotation shaft 22.

As described above, the center C3 of the rotation shaft 22 overlaps the center C1 of the first bearing hole 80a of the first jaw 32a. Therefore, the center C1 of the first bearing hole 80a is the rotation center of the first jaw 32a. Similarly, the center C3 of the rotation shaft 22 overlaps the center C2 of the second bearing hole 80b of the second jaw 32b. Therefore, the center C2 of the second bearing hole 80b is the rotation center of the second jaw 32b.

In this case, rotation directions of the first jaw 32a and the second jaw 32b are directions in which the first pinching portion 50a and the second pinching portion 50b approach each other. That is, the first jaw 32a and the second jaw 32b rotate in the direction in which they are closed. With this rotation, the first sliding contact surface 78a of the first protrusion 72a on the first proximal end portion 38a comes into sliding contact with the second flat surface 70b of the second proximal end portion 38b, and the second sliding contact surface 78b of the second protrusion 72b on the second proximal end portion 38b comes into sliding contact with the first flat surface 70a of the first proximal end portion 38a.

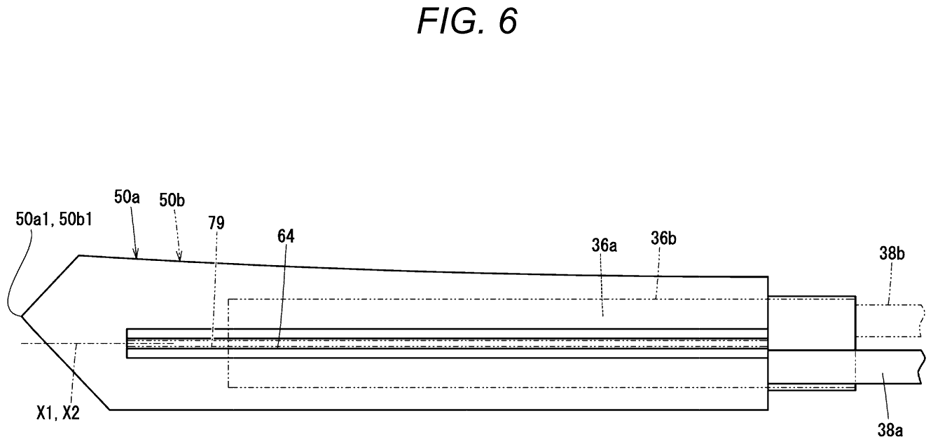

With the rotation described above, the first jaw 32a and the second jaw 32b are changed from the open state to the closed state, as illustrated in FIG. 5. Along with this change, the branch vessel BV (see FIG. 7) is pinched between the first pinching portion 50a and the second pinching portion 50b. The pair of side walls 61 of the spacer 60, which is exposed from the first slit 58a, comes into contact with the second electrode 54b. With this contact, the first electrode 54a and the second electrode 54b are slightly separated away from each other. In addition, the first cutter groove 64 and the second cutter groove 79 face each other as illustrated in FIG. 6.

Next, the operator operates the energization switch provided on the proximal end portion of the second cylindrical body 12. Thus, a high-frequency current is supplied to the first electrode 54a and the second electrode 54b. As a result, the branch vessel BV pinched between the first pinching portion 50a and the second pinching portion 50b is cauterized for hemostasis.

Next, the operator operates the cutter operation unit described above. At this time, the harvesting tool 30 is maintained in the closed state. Accordingly, the cutter 90 advances in the cutter space 94 illustrated in FIG. 4 and is inserted into the first cutter groove 64 and the second cutter groove 79. The cutter 90 further advances along the first cutter groove 64 and the second cutter groove 79 as illustrated in FIG. 7, and cuts the branch vessel BV pinched between the first pinching portion 50a and the second pinching portion 50b. If necessary, the medical device 10 may be further advanced in the body of the patient to cut another branch vessel or exfoliate surrounding tissues.

After the cutting of the branch vessel in the desired region is completed, the medical device 10 and the imaging device are withdrawn from the body of the patient. Thus, the main operation of the vessel harvesting is completed.

The present embodiment provides the following effects.

The medical device 10 includes a first jaw 32a and a second jaw 32b that are openable and closable by rotation and pinch a blood vessel when in a closed state. The first jaw 32a has a first pinching portion 50a and a first proximal end portion 38a connected to the proximal side of the first pinching portion 50a. The second jaw 32b has a second pinching portion 50b that pinches the blood vessel together with the first pinching portion 50a, and a second proximal end portion 38b connected to the proximal side of the second pinching portion 50b.

The first proximal end portion 38a and the second proximal end portion 38b have a first protrusion 72a and a second protrusion 72b, respectively. When the first jaw 32a and the second jaw 32b are in the closed state, the first protrusion 72a protrudes toward the second proximal end portion 38b, and the second protrusion 72b protrudes toward the first proximal end portion 38a. The first protrusion 72a has a first sliding contact surface 78a that comes into sliding contact with the second proximal end portion 38b when the first jaw 32a and the second jaw 32b are closed, and the second protrusion 72b has a second sliding contact surface 78b that comes into sliding contact with the first proximal end portion 38a when the first jaw 32a and the second jaw 32b are closed.

With this configuration, when the first jaw 32a and the second jaw 32b are closed, a state is formed in which the first sliding contact surface 78a of the first protrusion 72a comes into contact with the second proximal end portion 38b and the second sliding contact surface 78b of the second protrusion 72b comes into contact with the first proximal end portion 38a. Therefore, when the first jaw 32a and the second jaw 32b are in the closed state, it is easy to set the first axis X1 of the first pinching portion 50a and the second axis X2 of the second pinching portion 50b to be parallel to and face each other as illustrated in FIG. 6. In other words, when the first jaw 32a and the second jaw 32b are in the closed state, it is possible to suppress axial deviation between the first pinching portion 50a and the second pinching portion 50b. Therefore, it is possible to satisfactorily hold the blood vessel by the first pinching portion 50a and the second pinching portion 50b.

The first sliding contact surface 78a and the second sliding contact surface 78b may be in contact with the second proximal end portion 38b and the first proximal end portion 38a, respectively, even when the first jaw 32a and the second jaw 32b are opened.

The first proximal end portion 38a and the second proximal end portion 38b respectively have a first flat surface 70a and a second flat surface 70b facing each other when the first jaw 32a and the second jaw 32b are in the closed state. The first protrusion 72a protrudes from the first flat surface 70a toward the second flat surface 70b, and the second protrusion 72b protrudes from the second flat surface 70b toward the first flat surface 70a.

In this configuration, when the first jaw 32a and the second jaw 32b are closed, the first flat surface 70a and the second flat surface 70b gradually face each other, and the overlapping area in the side view as illustrated in FIG. 5 increases. Therefore, the first sliding contact surface 78a provided on the first protrusion 72a easily comes into contact with the second flat surface 70b (second proximal end portion 38b), and the second sliding contact surface 78b of the second protrusion 72b easily comes into contact with the first flat surface 70a (first proximal end portion 38a).

The first pinching portion 50a has a first cutter groove 64 extending along the direction of the axis of the first pinching portion 50a, and the second pinching portion 50b has a second cutter groove 79 extending along the direction of the axis of the second pinching portion 50b. The cutter 90 constituting the medical device 10 advances toward the distal ends of the first jaw 32a and the second jaw 32b in the closed state of the first jaw 32a and the second jaw 32b, and is inserted into the first cutter groove 64 and the second cutter groove 79. The cutter 90 further advances toward the distal ends of the first jaw 32a and the second jaw 32b along the first cutter groove 64 and the second cutter groove 79.

In this manner, the cutter 90 moves from the proximal end to the distal end along the first cutter groove 64 and the second cutter groove 79, and cuts the blood vessel. Here, as described above, axial deviation between the first pinching portion 50a and the second pinching portion 50b is suppressed (see FIG. 6). Therefore, when the first jaw 32a and the second jaw 32b are in the closed state, relative positional misalignment between the first cutter groove 64 and the second cutter groove 79 is unlikely to occur. Therefore, the cutter 90 is easily inserted into the first cutter groove 64 and the second cutter groove 79, and easily moves along the first cutter groove 64 and the second cutter groove 79.

The first protrusion 72a has a first linear portion 74a extending along the direction of axis of the first proximal end portion 38a, and the second protrusion 72b has a second linear portion 74b extending along the direction of axis of the second proximal end portion 38b. As illustrated in FIG. 5, when the first jaw 32a and the second jaw 32b are in the closed state, the first linear portion 74a and the second linear portion 74b are positioned parallel to each other.

When the cutter 90 moves in the closed state of the first jaw 32a and the second jaw 32b, the cutter 90 is prevented from interfering with the first linear portion 74a and the second linear portion 74b because the first linear portion 74a and the second linear portion 74b are parallel to each other. Therefore, even when the first jaw 32a and the second jaw 32b are in the closed state, the cutter 90 easily moves.

The first linear portion 74a is located proximal to the rotation center of the first jaw 32a, and the second linear portion 74b is located proximal to the rotation center of the second jaw 32b.

Due to the first linear portion 74a and the second linear portion 74b being positioned as described above, when the first jaw 32a and the second jaw 32b are changed from the open state to the closed state, the first proximal end portion 38a can be supported by the second sliding contact surface 78b of the second linear portion 74b while the second proximal end portion 38b can be supported by the first sliding contact surface 78a of the first linear portion 74a. Therefore, it is possible to further suppress the axial deviation between the first pinching portion 50a and the second pinching portion 50b.

The first protrusion 72a has a first inclined portion 76a connected to the distal side of the first linear portion 74a, and the second protrusion 72b has a second inclined portion 76b connected to the distal side of the second linear portion 74b. When the first jaw 32a and the second jaw 32b are in the open state, the first inclined portion 76a and the second inclined portion 76b are inclined in a direction not interfering with the cutter 90.

As illustrated in FIG. 8, in the open state of the first jaw 32a and the second jaw 32b, the first inclined portion 76a and the second inclined portion 76b serve as back clearance portions that do not interfere with the distal end of the cutter 90. In other words, even if the first jaw 32a and the second jaw 32b are in the open state when the cutter 90 is at the most retracted position, the first protrusion 72a and the second protrusion 72b are prevented from interfering with the cutter 90. As illustrated in FIGS. 1 and 2, even when the cutter 90 moves in the open state of the first jaw 32a and the second jaw 32b, the first protrusion 72a or the second protrusion 72b is prevented from interfering with the cutter 90.

Moreover, when the first jaw 32a and the second jaw 32b are changed from the open state to the closed state, the first inclined portion 76a and the first sliding contact surface 78a of the first linear portion 74a come into sliding contact with the second proximal end portion 38b, and the second inclined portion 76b and the second sliding contact surface 78b of the second linear portion 74b come into sliding contact with the first proximal end portion 38a. That is, in this case, the sliding contact area of the first sliding contact surface 78a and the second sliding contact surface 78b are larger than that in a case where the first protrusion 72a is constituted only by the first linear portion 74a obtained by cutting off the first inclined portion 76a and the second protrusion 72b is constituted only by the second linear portion 74b obtained by cutting off the second inclined portion 76b. Therefore, when the first jaw 32a and the second jaw 32b are changed from the open state to the closed state, it is possible to further suppress the axial deviation between the first pinching portion 50a and the second pinching portion 50b.

The first proximal end portion 38a has a first extension-side end portion 40a and a first non-extension-side end portion 42a, and the second proximal end portion 38b has a second extension-side end portion 40b and a second non-extension-side end portion 42b. Here, the first extension-side end portion 40a is an end portion having a distal end from which the first pinching portion 50a extends in the distal direction. The first non-extension-side end portion 42a is an end portion opposite to the first extension-side end portion 40a with reference to a first parallel line L1 passing through the rotation center of the first jaw 32a and parallel to the axis of the first jaw 32a. The second extension-side end portion 40b is an end portion having a distal end from which the second pinching portion 50b extends in the distal direction. The second non-extension-side end portion 42b is an end portion opposite to the second extension-side end portion 40b with reference to a second parallel line L2 passing through the rotation center of the second jaw 32b and parallel to the axis of the second jaw 32b. In this configuration, the first protrusion 72a is located at the first non-extension-side end portion 42a, and the second protrusion 72b is located at the second non-extension-side end portion 42b.

By providing the first protrusion 72a and the second protrusion 72b as described above, the first proximal end portion 38a can be supported by the second sliding contact surface 78b while the second proximal end portion 38b is supported by the first sliding contact surface 78a when the first jaw 32a and the second jaw 32b are changed from the open state to the closed state. Further, when the first jaw 32a and the second jaw 32b are in the open state, the first inclined portion 76a and the second inclined portion 76b can be prevented from interfering with the cutter 90.

Note that the present invention is not limited to the above disclosure and can take various configurations without departing from the gist of the present invention. For example, in the embodiment described above, a blood vessel that is an example of a luminal organ is described as an example of biological tissue, but an object to be harvested may be biological tissue other than a blood vessel as described above. The biological tissue includes a biological organ. The biological organ includes a solid organ, a luminal organ, or the like. Examples of the luminal organ other than the blood vessel include a digestive tract, a ureter, a trachea, and a cervical canal.

What is claimed is:

1. A medical device comprising a first jaw and a second jaw that are openable and closable by rotation and pinch biological tissue when in a closed state, wherein
the first jaw has a first pinching portion and a first proximal end portion connected to a proximal side of the first pinching portion,
the second jaw has a second pinching portion that pinches the biological tissue together with the first pinching portion, and a second proximal end portion connected to a proximal side of the second pinching portion,
the first proximal end portion and the second proximal end portion have a first protrusion and a second protrusion, respectively, the first protrusion protruding toward the second proximal end portion, and the second protrusion protruding toward the first proximal end portion, when the first jaw and the second jaw are in the closed state,
the first protrusion has a first sliding contact surface that comes into sliding contact with the second proximal end portion when the first jaw and the second jaw are closed, and the second protrusion has a second sliding contact surface that comes into sliding contact with the first proximal end portion when the first jaw and the second jaw are closed.

2. The medical device according to claim 1, wherein the first proximal end portion and the second proximal end portion respectively have a first flat surface and a second flat surface facing each other when the first jaw and the second jaw are in the closed state, the first protrusion protrudes from the first flat surface toward the second flat surface, and the second protrusion protrudes from the second flat surface toward the first flat surface.

3. The medical device according to claim 1, wherein the first pinching portion has a first cutter groove extending along a direction of an axis of the first pinching portion, and the second pinching portion has a second cutter groove extending along a direction of an axis of the second pinching portion,
the medical device further comprising a cutter that, when the first jaw and the second jaw are in the closed state, advances toward distal ends of the first jaw and the second jaw to be inserted into the first cutter groove and the second cutter groove and further advances toward the distal ends of the first jaw and the second jaw along the first cutter groove and the second cutter groove.

4. The medical device according to claim 3, wherein the first protrusion has a first linear portion extending along a direction of an axis of the first proximal end portion, and the second protrusion has a second linear portion extending along a direction of an axis of the second proximal end portion,
the first linear portion and the second linear portion being positioned parallel to each other, when the first jaw and the second jaw are in the closed state.

5. The medical device according to claim 4, wherein the first linear portion is located proximal to a rotation center of the first jaw, and the second linear portion is located proximal to a rotation center of the second jaw.

6. The medical device according to claim 4, wherein the first protrusion has a first inclined portion connected to a distal side of the first linear portion, and the second protrusion has a second inclined portion connected to a distal side of the second linear portion,
the first inclined portion and the second inclined portion being inclined in a direction not interfering with the cutter when the first jaw and the second jaw are in an open state.

7. The medical device according to claim 6, wherein the first proximal end portion has a first extension-side end portion that is an end portion having a distal end from which the first pinching portion extends in a distal direction, and a first non-extension-side end portion that is an end portion opposite to the first extension-side end portion with reference to a first parallel line passing through a rotation center of the first jaw and parallel to an axis of the first jaw, the second proximal end portion has a second extension-side end portion that is an end portion having a distal end from which the second pinching portion extends in the distal direction, and a second non-extension-side end portion that is an end portion opposite to the second extension-side end portion with reference to a second parallel line passing through a rotation center of the second jaw and parallel to an axis of the second jaw,
the first protrusion is located at the first non-extension-side end portion, and the second protrusion is located at the second non-extension-side end portion.

8. The medical device according to claim 1, wherein the first proximal end portion has a first extension-side end portion that is an end portion having a distal end from which the first pinching portion extends in a distal direction, and a first non-extension-side end portion that is an end portion opposite to the first extension-side end portion with reference to a first parallel line passing through a rotation center of the first jaw and parallel to an axis of the first jaw, the second proximal end portion has a second extension-side end portion that is an end portion having a distal end from which the second pinching portion extends in the distal direction, and a second non-extension-side end portion that is an end portion opposite to the second extension-side end portion with reference to a second parallel line passing through a rotation center of the second jaw and parallel to an axis of the second jaw,
the first protrusion is located at the first non-extension-side end portion, and the second protrusion is located at the second non-extension-side end portion.

9. A medical device comprising a first jaw and a second jaw that are openable and closable by rotation and pinch biological tissue when in a closed state, wherein
the first jaw has a first pinching portion and a first proximal end portion connected to a proximal side of the first pinching portion,
the second jaw has a second pinching portion that pinches the biological tissue together with the first pinching portion, and a second proximal end portion connected to a proximal side of the second pinching portion,
the first proximal end portion and the second proximal end portion have a first protrusion and a second protrusion, respectively, the first protrusion protruding toward the second proximal end portion, and the second protrusion protruding toward the first proximal end portion, when the first jaw and the second jaw are in the closed state,
the first protrusion has a first sliding contact surface that comes into sliding contact with the second proximal end portion when the first jaw and the second jaw are closed, and the second protrusion has a second sliding contact surface that comes into sliding contact with the first proximal end portion when the first jaw and the second jaw are closed;
wherein the first proximal end portion and the second proximal end portion respectively have a first flat surface and a second flat surface facing each other when the first jaw and the second jaw are in the closed state, the first protrusion protrudes from the first flat surface toward the second flat surface, and the second protrusion protrudes from the second flat surface toward the first flat surface; and
wherein the first pinching portion has a first cutter groove extending along a direction of an axis of the first pinching portion, and the second pinching portion has a second cutter groove extending along a direction of an axis of the second pinching portion;
the medical device further comprising a cutter that, when the first jaw and the second jaw are in the closed state, advances toward distal ends of the first jaw and the second jaw to be inserted into the first cutter groove and the second cutter groove and further advances toward the distal ends of the first jaw and the second jaw along the first cutter groove and the second cutter groove.

10. A medical device comprising a first jaw and a second jaw that are openable and closable by rotation and pinch biological tissue when in a closed state, wherein
the first jaw has a first pinching portion and a first proximal end portion connected to a proximal side of the first pinching portion,
the second jaw has a second pinching portion that pinches the biological tissue together with the first pinching portion, and a second proximal end portion connected to a proximal side of the second pinching portion,
the first proximal end portion and the second proximal end portion have a first protrusion and a second protrusion, respectively, the first protrusion protruding toward the second proximal end portion, and the second protrusion protruding toward the first proximal end portion, when the first jaw and the second jaw are in the closed state,
the first protrusion has a first sliding contact surface that comes into sliding contact with the second proximal end portion when the first jaw and the second jaw are closed, and the second protrusion has a second sliding contact surface that comes into sliding contact with the first proximal end portion when the first jaw and the second jaw are closed;
wherein the first proximal end portion and the second proximal end portion respectively have a first flat surface and a second flat surface facing each other when the first jaw and the second jaw are in the closed state, the first protrusion protrudes from the first flat surface toward the second flat surface, and the second protrusion protrudes from the second flat surface toward the first flat surface; and
wherein the first protrusion has a first linear portion extending along a direction of an axis of the first proximal end portion, and the second protrusion has a second linear portion extending along a direction of an axis of the second proximal end portion, and
the first linear portion and the second linear portion being positioned parallel to each other, when the first jaw and the second jaw are in the closed state.

11. The medical device according to claim 10, wherein the first linear portion is located proximal to a rotation center of the first jaw, and the second linear portion is located proximal to a rotation center of the second jaw.

12. The medical device according to claim 10, wherein the first pinching portion has a first cutter groove extending along a direction of an axis of the first pinching portion, and the second pinching portion has a second cutter groove extending along a direction of an axis of the second pinching portion,
the medical device further comprising a cutter that, when the first jaw and the second jaw are in the closed state, advances toward distal ends of the first jaw and the second jaw to be inserted into the first cutter groove and the second cutter groove and further advances toward the distal ends of the first jaw and the second jaw along the first cutter groove and the second cutter groove.

13. The medical device according to claim 12, wherein the first protrusion has a first inclined portion connected to a distal side of the first linear portion, and the second protrusion has a second inclined portion connected to a distal side of the second linear portion,
the first inclined portion and the second inclined portion being inclined in a direction not interfering with the cutter when the first jaw and the second jaw are in an open state.
